(19) United States
(12) Patent Application Publication
(10) Pub. No.: US 2014/0018755 A1
(43) Pub. Date: Jan. 16, 2014

(54) HYGIENIC SWAB

(71) Applicant: Karl Thews, Mannheim (DE)

(72) Inventor: Karl Thews, Mannheim (DE)

(21) Appl. No.: 14/007,361

(22) PCT Filed: Apr. 2, 2012
(86) PCT No.: PCT/EP2012/055992

§ 371 Date: Sep. 25, 2013

(30) Foreign Application Priority Data
Apr. 1, 2011 (DE) 10 2011 015 889.8

(51) Int. Cl. A61F 13/15

(52) U.S. Cl. CPC A61F 13/15252
USPC 604/364; 264/319

(57) ABSTRACT

A hygienic swab for the skin. It is an aim to provide a basic standard of hygiene for users of urinals and, for this purpose, to make available a hygiene article that can be readily disposed of in the basin of a urinal. The swab is formed from a shaped and dimensionally stable tablet having an absorbent structure, wherein at least part of the structure has at least one substance or a mixture of several different substances in the form of granules or grains.

### FIELD OF THE INVENTION

The invention relates to a hygienic swab for the skin for the manual dabbing of urine in the area of the urethral opening.

### BACKGROUND OF THE INVENTION

Absorbent articles such as toilet paper and moistened cloths are already known for the dabbing and cleaning of the skin. In particular, absorbent articles have been described in which cellulose or sugar crystals are used.

U.S. Pat. No. 6,281,407 A specifies absorbent articles in which fructose and glucose are also used as drying agent, the drying agent being located in a pocket.

U.S. 2007/219521 A specifies monosaccharides and disaccharides as intermediate compounds for synthetic polymers, such as cellulose fibers.

U.S. 2011/028928 A specifies a polysaccharide for absorption and gel formation, being located in a tissue.

EP 1934389 A1 specifies thermoplastic fibers for a hygiene article that are stable in water.

### SUMMARY OF THE INVENTION

The underlying problem of the invention is to provide a basic standard of hygiene for users of urinals and, for this purpose, to create a hygiene article that can be readily disposed of in the basin of a urinal.

The problem is solved according to the invention in that the swab is formed from a shaped and dimensionally stable tablet (1) having an absorbent structure, wherein at least part of the structure has at least one substance or a mixture of several different substances in the form of granules or grains.

Absorbent here has the meaning of being able to take up and retain liquid.

A structure here specifies a more or less dense mixture of grains and particles of solid substances, whose ingredients are in a primarily mechanical but not chemical interaction with each other. The structure of the invention is absorbent, has a tremendous capillary action, is dimensionally stable, and only decomposes upon uptake of more than around 20 ml of liquid, depending on the size or volume.

Such a swab in the form of a tablet, thanks to its attribute of first decomposing in water and then being dissolved in water, can also be disposed of without any problems even in a urinal, which usually has a reduced drain geometry and a reduced cross section of the sewage lines. There is no danger of clogging.

The danger of clogging of sewage lines with small cross section less than 80 mm that is associated with toilet paper or other known hygiene articles has had the consequence, since the advent of modern sewage collection systems, that no hygiene articles were available to the user of a urinal for drying the skin in the region of the urethral opening. This extremely unhygienic situation occurs especially in public toilet facilities, such as those in hotels, restaurants, schools, rest stops and other public toilets.

Urine is an initially germ-free and almost odorless bodily fluid that contains urea and uric acid, in addition to water. But the germ-free condition is not stable after leaving the bladder, either in the urethra or in other genital areas where urine collects, because the urine comes into contact here with oxygen and germs from the surroundings. In this no longer germ-free condition, a decomposition process begins, by which the odorless quality is lost after only two to three hours. The quantity of no longer germ-free urine that collects around the urethral opening in the urethra and other genital areas can amount to between 0.5 ml and 2 ml, or between 5 drops and 15 drops. The swab can be used to dab the liquid on the skin surface and around the urethral opening. But at the same time, due to the suction effect of the swab, urine is drawn out from the urethra and likewise taken up by the swab. After this, the user simply drops the swab into the basin.

The swab is primarily suited to the male gender, for which reason the swab has an elevated uptake capacity of as much as 4 ml. Even at maximum liquid uptake, the holding area remains dry and the swab remains dimensionally stable, without becoming totally wet in parts. Only when the swab is completely wet does it decompose entirely after a few minutes, at first forming a sediment and then dissolving for the most part into tiny pieces or entirely.

The sample embodiments, uses and areas of application are specified in the claims.

The substance or substances for the structure are preferably sugar (saccharose) and hydrophilic starch. Sugar (saccharose) serves here as a collective term for all household sugars that can be refined in granular or powder form in order to make such tablets. In particular, saccharose is used from sugar beets or sugar cane, which can be readily pressed, especially when the crystalline sugar is sifted in the form of refined sugar. Furthermore, sugar is a relatively favorable raw material that is available worldwide and, unlike paper, it can be produced in an environmentally friendly and sustainable manner.

By adding further substances, such as fragrance or bitters, a colorant, or some other substance, the tablet can be made unsuitable for any consumption.

The sugar or the granulate form with the starch a heterogeneous structure. The water-soluble (hydrophilic) starch according to the invention gives the surface of the tablet a smooth texture and at the same time helps during the pressing.

Another major benefit from the use of hydrophilic starch is its property of not clumping in combination with water but instead becoming homogeneously distributed in the water. The starch grains or particles are very small in comparison to the sugar grains, but do not themselves dissolve fully in the water, unlike the sugar.

The tablets can have any basic shape, such as square, cube, cylinder, cone or octahedron. Spheres and other combinations of such shapes can also be realized.

Thanks to the concave shape, it is possible to place, say, two tablets, arranged one on top of the other, against each other on a very small, almost pointlike surface. Since this surface at the same time is the surface on which the two swabs slide against each other when being removed from the dispensing device, the friction between the two tablets is reduced by the small surface.

The tablet is absorbent on the whole, so that in the case of very small tablets the situation may arise where liquid is suctioned beyond the absorption area into the holding area. In order to avoid all contact between the fingers and the liquid during manual use, it is arranged to isolate the holding area from the adsorption area. For this, a barrier layer can be produced by glazed sugar or by an additive at least inhibiting the capillary action. The barrier layer is not dimensionally stable upon decomposition of the tablet in water.

A major advantage for the manufacturing process to enhance the absorptivity is the increasing of the capillary effect between the individual sugar grains and the uptake capacity by adjusting the pressing force when manufacturing the shaped body as a function of the mean grain size of the sugar crystals and/or the size of the starch particles.

### BRIEF DESCRIPTION OF THE DRAWINGS

Further benefits and details of the invention are explained in the patent claims and in the specification and represented in the figures. There are shown:

FIG. 1a a side view of a convex curved tablet;

FIG. 1b a view from above of the tablet of FIG. 1a;

FIG. 2 a side view of a stack of tablets;

FIG. 3a a side view of a stack of tablets in a dispensing device;

FIG. 3b a view of a stack of tablets according to FIG. 3a, from the front;

FIG. 3c a view of a stack of tablets according to FIG. 3a, from above;

FIG. 4 a square-shaped tablet;

FIG. 5 a disk-shaped tablet;

FIG. 6 a cylinder-shaped tablet;

FIG. 7a a dispensing device filled with tablets according to FIG. 6 in a side view;

FIG. 7b the dispensing device of FIG. 7a in a view from the front.

### DETAILED DESCRIPTION OF THE INVENTION

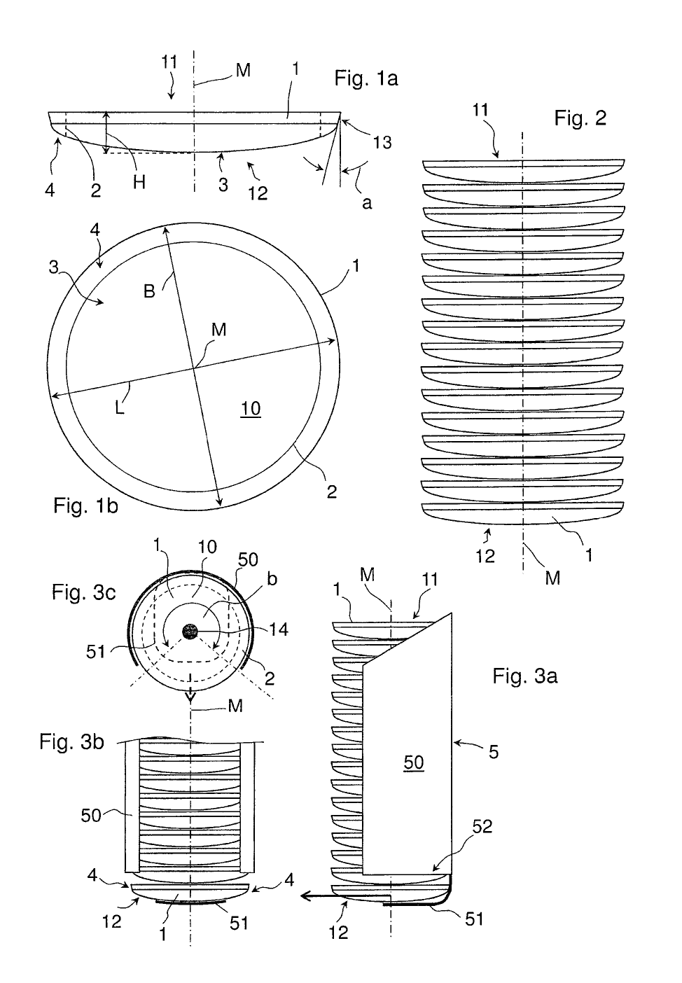

Swabs, such as are represented in FIGS. 1a to 7a, basically form a 3-dimensional tablet 1 with a length L, a width B and a height H. The surface 10 comprises the top side 11 and the bottom side 12. A portion of the overall surface 10 serves as an absorption area 3 to take up liquid. In the case of flat, rotationally symmetrical tablets 1, the absorption area 3 is preferably situated in the center of the tablet 1 and the holding areas 4 at the margin. In the case of rod-shaped tablets 1, the absorption area 3 is provided in the direction of the axis R on one side and the holding area 4 on the opposite side. Very simple configured swabs consist of a simple homogeneous structure pressed into a tablet 1, similar to a large sugar cube.

Preferably, the structure of the tablet 1 is formed from pressed granular-crystalline sugar, alone or in combination with water-soluble starch.

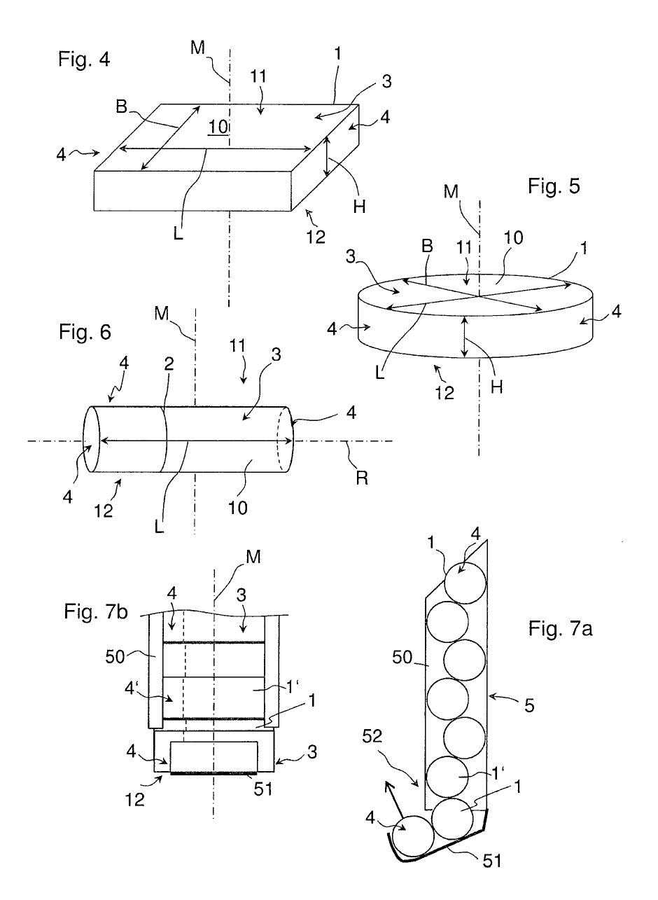

In FIG. 1a to 3c are shown sample embodiments of a tablet 1 that is rotationally symmetrical to a center axis M, with a concave top side 11 and a flat bottom side 12. These are more costly in terms of their external shape than simple tablets as shown in FIG. 4 et seq., but they have advantages in the manufacturing and the dispensing.

The tablet 1 per FIG. 1a to 3a has a bevel 13 encircling the center axis M with an angle a of 3°, so that the tablet 1 drops easily out from a pressing mold, not shown. Furthermore, the swab 1 as depicted in FIG. 1 has a barrier layer 2, by which the absorption area 3 is bounded off from a holding area 4 provided at the margin. The barrier layer 2 prevents the liquid taken up via the absorption area 3 from being suctioned into the holding area 4, so that the holding area 4 remains hygienically dry. The barrier layer 2 is made from a substance that is primarily water repellent or not very water permeable and can be easily pressed with the structure. The barrier layer 2 is a few tenths of a millimeter thick in the radial direction to the center axis M.

The tablets 1 are stacked on one another in the vertical direction as shown in FIG. 2 and inserted in this formation in a holder 50 of a dispensing device 5 as shown in FIGS. 3a to 3c. In the holder 50, the stack drops down by gravity as shown in FIG. 3a and is held by the shoulder 51 arranged beneath the holder 50. In FIG. 3c the shape of the holder 50 is shown, the tablet 1 being encircled in the circumferential direction to the center axis M by a sector with an angle b of around 270 degrees, ensuring a support in all radial directions to the center axis M.

In the lateral holding area 4, the bottom tablet 1 is free and not enclosed by the holder 50, as shown in FIG. 3b. The lower end 52 of the holder 50 is recessed in the upper direction relative to the lowest tablet 1 in the stack. Thus, the tablet 1 can be grasped manually in the holding area 4 and pulled out in the direction of the arrow shown in FIG. 3a from the dispensing device 5 and it is only radially supported in one direction, opposite the arrow shown.

Thanks to the flat and concave shape, the tablets 1 arranged one on top of the other only touch each other in a small circular area 14, arranged concentric to the center axis M. This reduces the friction between the tablets 1 and improves the sliding ability between the tablets 1. In particular, swabs 1 of pure sugar, without addition of starch, have a tendency to stick when kept in a humid environment, so that the tablets 1 are still easy to separate from each other thanks to the shaping and the small bearing surface 14 according to the invention.

The tablet 1 shown in FIG. 4 forms a square, similar to a large flat sugar cube. This simple shape favors the manufacturing process and a small package size. The square tablet 1, just like the previously described swab 1, is stacked one on top of another in the direction of the center axis M in a holder 50 (not shown). The mode of operation of the dispensing device 5 likewise corresponds to that described above. Furthermore, the tablet 1 of FIG. 4 has no barrier layer 2. Here, the holding area 4 is not isolated from the absorption area 3. It has been investigated that the swab 1 does not necessarily need a barrier layer 2, because a tablet 1 with a practical size corresponding approximately to 4 to 6 times the volume of an ordinary sugar cube can take up enormous quantities of liquid in its middle and thus in the absorption area 3 before the liquid makes it into the holding area 4 at the margin.

The tablet 1 shown in FIG. 5 is likewise rotationally symmetrical to the center axis M, but the top side 11 and the bottom side 12 are flat in shape. Here as well, thanks to the minimum size for a practical handling and the corresponding volume, one can do without a separate protection layer 3 between the absorption area 3 and the holding area 4.

In the tablet 1 shown in FIG. 6, the absorption area 3 is provided on the right side in the direction of the axis R and the holding area 4 on the opposite, left side. As with other rod-shaped objects, such as lipsticks or glue sticks, the tablet 1 is manually grasped at one end in the direction of the axis R after being removed from the dispensing device 5. For this, the structure has the necessary stability in all described compositions, especially the necessary bending strength along the axis R.

FIGS. 7a and 7b show a similar dispensing device 5 to that described above for disk-shaped tablets 1. The dispensing device 5 is loaded with swabs 1, as described in FIG. 6. The rod-shaped tablets 1 lie with their length L horizontally oriented one on top of another in the dispensing device 5. The dispensing device 5 is shortened at its lower end 52 relative to the shoulder 51, so that the lowest swab 1 lies on the shoulder 51, able to be grasped freely from above and from the side. The swab 1 is removed from the shoulder 51 by grasping it manually in the holding area 4. After this, the swab 1 is held in the holding area 4 and the absorption area 3 serves to take up liquid with its entire surface 10.

What is claimed is:

1. A hygienic swab for the skin, comprising: the swab formed from a tablet with a shaped, dimensionally stable, and absorbent structure; wherein the dimensionally stable structure decomposes in water.

2. The swab according to claim 1, wherein the structure is formed from one or more of sugar (saccharose), salt, cellulose and starch.

3. The swab according to claim 1, wherein additives are contained in the structure to improve the properties of the tablet in regard to
a) pressing, shaping, abrasion
b) dissolving and/or
c) skin toleration, skin care, color, fragrance.

4. The swab according to claim 1, wherein the structure or the substance has a crystalline shape and/or a grain size between 0.1 mm and 1 mm.

5. The swab according to claim 1, wherein the tablet has several areas with a same or different structure, wherein one area is configured as an absorption area for taking up liquids and the other area as a holding area.

6. The swab according to claim 5, wherein a barrier layer is provided between the holding area and the absorption area, separating the two areas from a liquid transport.

7. The swab according to claim 1, wherein a separate handle is arranged on the tablet.

8. The swab according to claim 1, wherein the tablet has a symmetrical or asymmetrical shape with a maximum length (L) between 3 cm and 8 cm, a maximum width (B) between 0.5 cm and 6 cm and a maximum height (H) between 2 mm and 18 mm.

9. The swab according to claim 1, wherein the tablet has a weight between 5 grams and 30 grams.

10. The swab according to claim 1, wherein the tablet is rotationally symmetrical with respect to a center axis (M) and has a flat or convex curved top side and an opposite flat or convex curved bottom side.

11. The swab according to claim 1, it wherein the swab has a surface with a surface quality that corresponds to a mean peak to valley height between 3 μm and 22 μm and the surface is free of dust and/or crumbs.

12. A system comprising: a swab according to claim 1 and a dispensing device for keeping and dispensing several swabs stacked one on top of the other in a direction of a center axis (M) for placement in a bathroom.

13. A method for manufacturing a swab by comprising a tablet with a shaped, dimensionally stable and absorbent structure, comprising the following steps:
a) mixing the structure into a homogeneous structure
b) adding between 2 wt. % and 10 wt. % of water, and
c) mechanical shaping and/or pressing of individual tablets one after the other or several tablets at the same time.

14. A method for using the swab according to claim 1, comprising the step of disposing of the swab in a urinal.

15. A method comprising the steps of: using a sugar cube as a hygienic swab for manual dabbing and taking up of urine from a genital organ.

16. The swab according to claim 2, wherein additives are contained in the structure to improve the properties of the tablet in regard to
a) pressing, shaping, abrasion
b) dissolving and/or
c) skin toleration, skin care, color, fragrance; and
wherein the structure or the substance has a crystalline shape and/or a grain size between 0.1 mm and 1 mm.

17. The swab according to claim 16, wherein the tablet has several areas with a same or different structure, wherein one area is configured as an absorption area for taking up liquids and the other area as a holding area, and wherein a barrier layer is provided between the holding area and the absorption area, separating the two areas from a liquid transport.

18. The swab according to claim 17, wherein a separate handle is arranged on the tablet, and wherein the tablet has a symmetrical or asymmetrical shape with a maximum length (L) between 3 cm and 8 cm, a maximum width (B) between 0.5 cm and 6 cm and a maximum height (H) between 2 mm and 18 mm.

19. The swab according to claim 18, wherein the tablet has a weight between 10 grams and 16 grams, and wherein the tablet is rotationally symmetrical with respect to a center axis (M) and has a flat or convex curved top side and an opposite flat or convex curved bottom side.

20. The swab according to claim 19, wherein the swab has a surface with a surface quality that corresponds to a mean peak to valley height between 3 μm and 22 μm and the surface is free of dust and/or crumbs.
